(19) United States
(12) Patent Application Publication
(10) Pub. No.: US 2025/0285553 A1
(43) Pub. Date: Sep. 11, 2025

(54) Mobile Application for Interactive Osteopathic Medical Education: A Novel Approach to Osteopathic Manipulative Treatment (OMT) Instruction

(71) Applicant: Lindsey Callier, Atlanta, GA (US)

(72) Inventor: Lindsey Callier, Atlanta, GA (US)

(21) Appl. No.: 18/595,476

(22) Filed: Mar. 5, 2024

(51) Int. Cl. G09B 19/00; G06Q 50/00; G16H 80/00; H04L 67/00

(52) U.S. Cl. CPC G09B 19/00; G06Q 50/01; G16H 80/00; H04L 67/34

(57) ABSTRACT

Methods, storage media and systems for integrating osteopathic manipulative medicine (OMM) education into a medical school curriculum are disclosed. Some embodiments may include: developing an interactive mobile application including OMM teaching content; providing access to the mobile application to a plurality of medical students, collecting baseline OMM competency data from the plurality of medical students before providing access, collecting post-intervention OMM competency data after a defined period of access to the mobile application and evaluating an effectiveness of the mobile application by analyzing the baseline and post-intervention competency data.

### FIELD OF THE DISCLOSURE

The present disclosure relates to methods, storage media and systems for integrating osteopathic manipulative medicine education into a medical school curriculum.

### BACKGROUND

Osteopathic Manipulative Medicine (OMM) is a vital component of holistic patient care which focuses on the interrelationship between the musculoskeletal system and overall health. However, OMM education in medical schools faces significant challenges regarding effective integration amid overloaded curricula. Most medical students receive only basic exposure in preclinical years, leading to knowledge gaps when entering clinical practice. Consequently, utilization of OMM by students in clinical rotations and physicians in practice remains suboptimal despite the demonstrated patient benefits of decreased pain, improved immune response, and reduced recovery times.

Integrating OMM into the clinical phase of medical training has shown substantial promise in bridging knowledge to practice. Accelerated OMM courses and workshops during third and fourth year rotations have successfully increased competency and willingness to employ techniques. Additionally, hands-on courses during residency programs help reinforce concepts learned previously. However, heavy clinical workloads constrain the expansion of these interventions across institutions. Supplemental OMM teaching tools for independent mobile learning can address the barriers of limited teaching faculty and packed curricula.

The rapid adoption of mobile devices among medical students and clinicians has created opportunities for technology-enhanced delivery of point-of-care education. The development of a comprehensive mobile app to augment OMM integration aims to enhance knowledge, build confidence in practical skills, and facilitate clinical application. Effective app design and evaluation through robust research is essential to support adoption among students and cultivate enthusiasm for osteopathic principles. Demonstrating the app's ability to assist overburdened students to independently reinforce their competency in OMM techniques would motivate broader curricular adoption.

It is therefore an object of the current invention to capitalize on mobile learning innovations to supplement OMM education in order to directly tackle existing integration obstacles and strengthen the future osteopathic workforce through positive learning experiences. It is a further object to refine pedagogical understanding of optimal app-enabled approaches as this remains key to seizing the promising avenue of mobile apps for reinvigorating osteopathic identity in future physicians. Specifically, this invention aims to develop and validate a specialized mobile app containing comprehensive OMM teaching content and hands-on practice to enhance students' knowledge, confidence, and clinical application of techniques. Quantitatively and qualitatively assessing the app's impact on competency and utilization will help optimize educational strategies to perpetuate osteopathic principles through technology.

### SUMMARY

One aspect of the present disclosure relates to a method for integrating osteopathic manipulative medicine education into a medical school curriculum. The method may include developing an interactive mobile application including OMM teaching content; providing access to the mobile application to a plurality of medical students. The method may include collecting baseline OMM competency data from the plurality of medical students before providing access. The method may include collecting post-intervention OMM competency data after a defined period of access to the mobile application. The method may include evaluating an effectiveness of the mobile application by analyzing the baseline and post-intervention competency data.

Another aspect of the present disclosure relates to a computer-readable storage medium for integrating osteopathic manipulative medicine (OMM) education into a medical school curriculum. In some embodiments, the computer-readable storage medium may include instructions being executable by one or more processors to develop an interactive mobile application including OMM teaching content; providing access to the mobile application to a plurality of medical students. In some embodiments, the computer-readable storage medium may include instructions being executable by one or more processors to collect baseline OMM competency data from the plurality of medical students before providing access. In some embodiments, the computer-readable storage medium may include instructions being executable by one or more processors to collect post-intervention OMM competency data after a defined period of access to the mobile application. In some embodiments, the computer-readable storage medium may include instructions being executable by one or more processors to evaluate an effectiveness of the mobile application by analyzing the baseline and post-intervention competency data.

Another aspect of the present disclosure relates to a system for integrating osteopathic manipulative medicine education into a medical school curriculum. The system may include one or more hardware processors configured by machine-readable instructions for integrating osteopathic manipulative medicine education into a medical school curriculum. The machine-readable instructions may be configured to develop an interactive mobile application including OMM teaching content; providing access to the mobile application to a plurality of medical students. The machine-readable instructions may be configured to collect baseline OMM competency data from the plurality of medical students before providing access. The machine-readable instructions may be configured to collect post-intervention OMM competency data after a defined period of access to the mobile application. The machine-readable instructions may be configured to evaluate an effectiveness of the mobile application by analyzing the baseline and post-intervention competency data.

### BRIEF DESCRIPTION OF THE DRAWINGS

The novel features believed to define the illustrative embodiments are detailed in the appended claims. To fully comprehend these embodiments, along with their preferred usage, objectives, and detailed descriptions, one should refer to the comprehensive description of the one or more examples of these embodiments, as provided in this disclosure. This understanding is further enhanced when considered alongside the accompanying drawings, wherein:

FIG. 1 illustrates a system configured for integrating osteopathic manipulative medicine (OMM) education into a medical school curriculum.

FIG. 2 illustrates a method for integrating osteopathic manipulative medicine (OMM) education into a medical school curriculum.

### DETAILED DESCRIPTION

The detailed description of the preferred embodiments of this invention for an osteopathic manipulative medicine educational mobile application is presented here, with references to the accompanying drawings. The specific terms and words used in both the description and the claims of this invention should not be confined to their ordinary or dictionary definitions. Instead, their interpretation should align with the meanings and concepts relevant to the mobile application invention, reflecting the inventor(s)' ability to define terms uniquely to best convey the invention.

It should be noted that the embodiments of the mobile application invention illustrated and discussed in this document represent preferred examples, and does not intent to restrict the technical essence or boundaries of the invention. Therefore, it is important to acknowledge that various alterations and adaptations can be made to the osteopathic manipulative medicine educational mobile application, which are still within its spirit and scope. For example, additional multimedia formats, interactive elements, integrated sensors, and connectivity with other platforms may be incorporated over time to further enhance the learning experience, while retaining alignment with the core inventive concept of a technology-driven supplement to standard curricula.

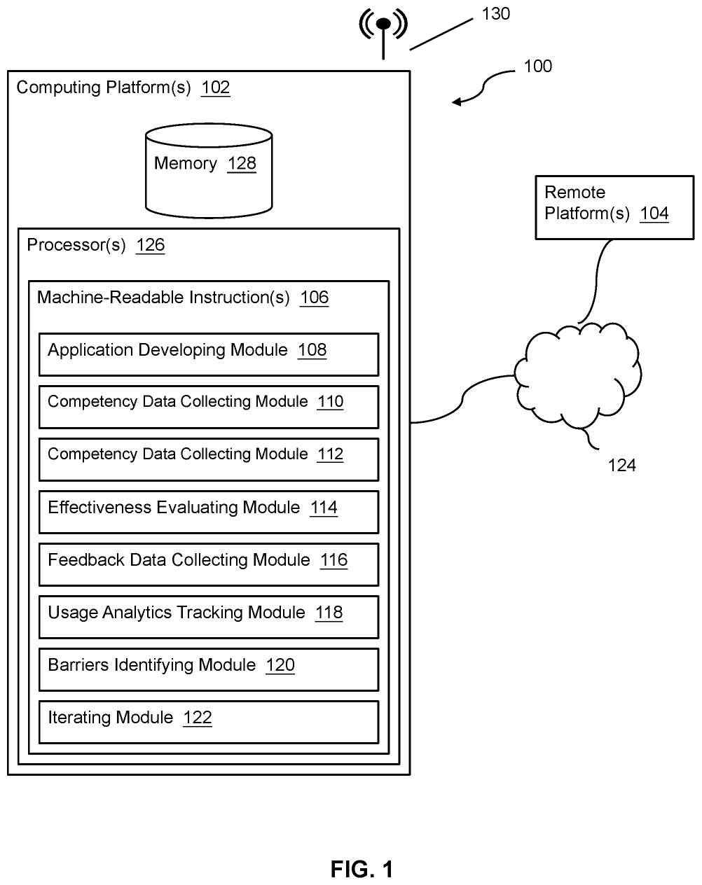

FIG. 1 illustrates a system configured for integrating osteopathic manipulative medicine (OMM) education into a medical school curriculum, in accordance with one or more embodiments. In some cases, system 100 may include one or more computing platforms 102. The one or more remote computing platforms 102 may be communicably coupled with one or more remote platforms 104. In some cases, users may access the system 100 via remote platform(s) 104.

The one or more computing platforms 102 may be configured by machine-readable instructions 106. Machine-readable instructions 106 may include modules. The modules may be implemented as one or more of functional logic, hardware logic, electronic circuitry, software modules, and the like. The modules may include one or more of application developing module 108, competency data collecting module 110, competency data collecting module 112, effectiveness evaluating module 114, feedback data collecting module 116, usage analytics tracking module 118, barriers identifying module 120, iterating module 122, and/or other modules.

Application developing module 108 may be configured to develop an interactive mobile application including OMM teaching content; providing access to the mobile application to a plurality of medical students. Competency data collecting module 110 may be configured to collect baseline OMM competency data from the plurality of medical students before providing access. Competency data collecting module 112 may be configured to collect post-intervention OMM competency data after a defined period of access to the mobile application. Effectiveness evaluating module 114 may be configured to evaluate an effectiveness of the mobile application by analyzing the baseline and post-intervention competency data.

In some cases, the OMM teaching content comprises instructional videos, tutorials, case studies, and reference materials on OMM principles and techniques and the baseline and post-intervention competency data comprises surveys evaluating medical students' knowledge, confidence levels, perceptions, and utilization rates related to OMM.

Feedback data collecting module 116 may be configured to collect qualitative feedback data from medical students through interviews and focus groups regarding experiences using the mobile application.

Usage analytics tracking module 118 may be configured to track usage analytics of interactions with the mobile application by the plurality of medical students.

In some cases, the plurality of medical students comprise third and fourth year students undertaking clinical rotations and evaluating the effectiveness comprises performing statistical analysis to compare baseline and post-intervention quantitative competency data.

Barriers identifying module 120 may be configured to identify barriers and facilitators to adoption of the mobile application based on qualitative feedback data.

Iterating module 122 may be configured to iterate development of the mobile application based on the analyzed effectiveness and qualitative feedback data.

In some cases, the mobile application may be configured to integrate with an electronic medical records system to surface contextually relevant OMM teaching content.

In some cases, the one or more computing platforms 102, may be communicatively coupled to the remote platform(s) 104. In some cases, the communicative coupling may include communicative coupling through a networked environment 124. The networked environment 124 may be a radio access network, such as LTE or 5G, a local area network (LAN), a wide area network (WAN) such as the Internet, or wireless LAN (WLAN), for example. It will be appreciated that this is not intended to be limiting, and that the scope of this disclosure includes implementations in which one or more computing platforms 102 and remote platform(s) 104 may be operatively linked via some other communication coupling. The one or more one or more computing platforms 102 may be configured to communicate with the networked environment 124 via wireless or wired connections. In addition, in an embodiment, the one or more computing platforms 102 may be configured to communicate directly with each other via wireless or wired connections. Examples of one or more computing platforms 102 may include, but is not limited to, smartphones, wearable devices, tablets, laptop computers, desktop computers, Internet of Things (IoT) device, or other mobile or stationary devices. In an embodiment, system 100 may also include one or more hosts or servers, such as the one or more remote platforms 104 connected to the networked environment 124 through wireless or wired connections. According to one embodiment, remote platforms 104 may be implemented in or function as base stations (which may also be referred to as Node Bs or evolved Node Bs (eNBs)). In other embodiments, remote platforms 104 may include web servers, mail servers, application servers, etc. According to certain embodiments, remote platforms 104 may be standalone servers, networked servers, or an array of servers.

The one or more computing platforms 102 may include one or more processors 126 for processing information and executing instructions or operations. One or more processors 126 may be any type of general or specific purpose processor. In some cases, multiple processors 126 may be utilized according to other embodiments. In fact, the one or more processors 126 may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. In some cases, the one or more processors 126 may be remote from the one or more computing platforms 102, such as disposed within a remote platform like the one or more remote platforms 126 of FIG. 1.

The one or more processors 126 may perform functions associated with the operation of system 100 which may include, for example, precoding of antenna gain/phase parameters, encoding and decoding of individual bits forming a communication message, formatting of information, and overall control of the one or more computing platforms 102, including processes related to management of communication resources.

The one or more computing platforms 102 may further include or be coupled to a memory 128 (internal or external), which may be coupled to one or more processors 126, for storing information and instructions that may be executed by one or more processors 126. Memory 128 may be one or more memories and of any type suitable to the local application environment, and may be implemented using any suitable volatile or nonvolatile data storage technology such as a semiconductor-based memory device, a magnetic memory device and system, an optical memory device and system, fixed memory, and removable memory. For example, memory 128 can consist of any combination of random access memory (RAM), read only memory (ROM), static storage such as a magnetic or optical disk, hard disk drive (HDD), or any other type of non-transitory machine or computer readable media. The instructions stored in memory 128 may include program instructions or computer program code that, when executed by one or more processors 126, enable the one or more computing platforms 102 to perform tasks as described herein.

In some embodiments, one or more computing platforms 102 may also include or be coupled to one or more antennas 130 for transmitting and receiving signals and/or data to and from one or more computing platforms 102. The one or more antennas 130 may be configured to communicate via, for example, a plurality of radio interfaces that may be coupled to the one or more antennas 130. The radio interfaces may correspond to a plurality of radio access technologies including one or more of LTE, 5G, WLAN, Bluetooth, near field communication (NFC), radio frequency identifier (RFID), ultrawideband (UWB), and the like. The radio interface may include components, such as filters, converters (for example, digital-to-analog converters and the like), mappers, a Fast Fourier Transform (FFT) module, and the like, to generate symbols for a transmission via one or more downlinks and to receive symbols (for example, via an uplink).

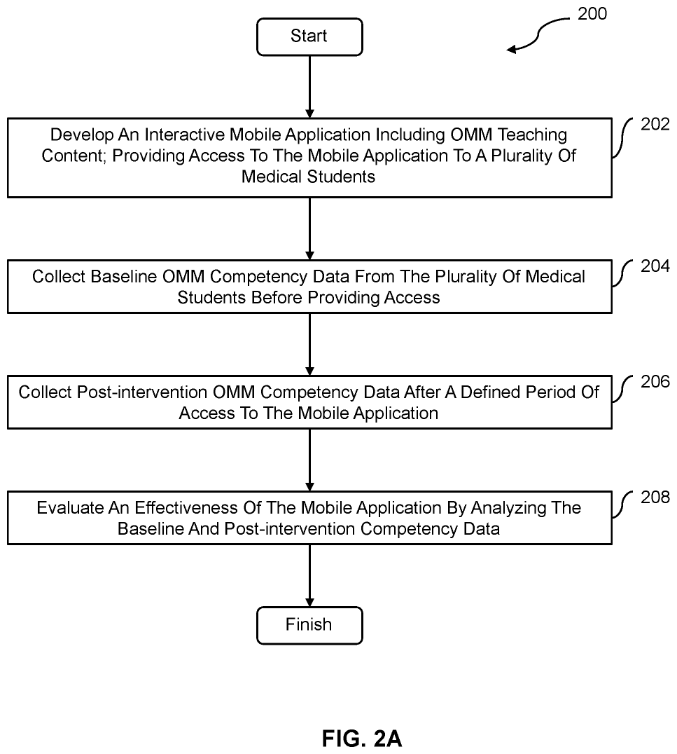

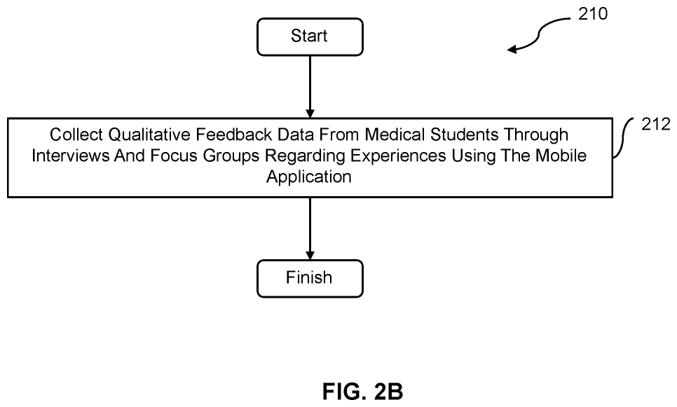

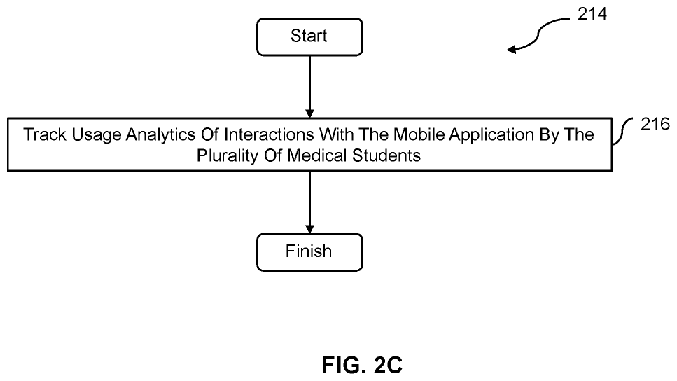

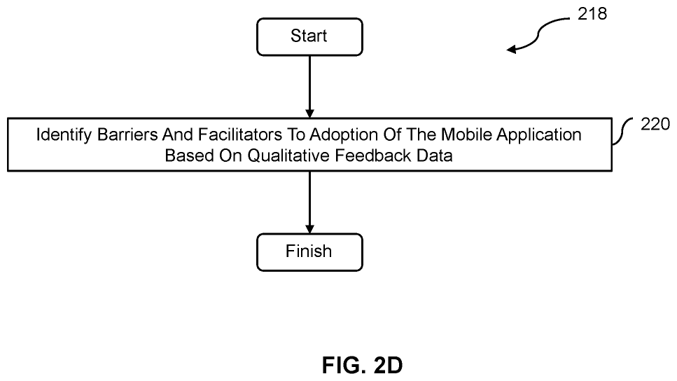

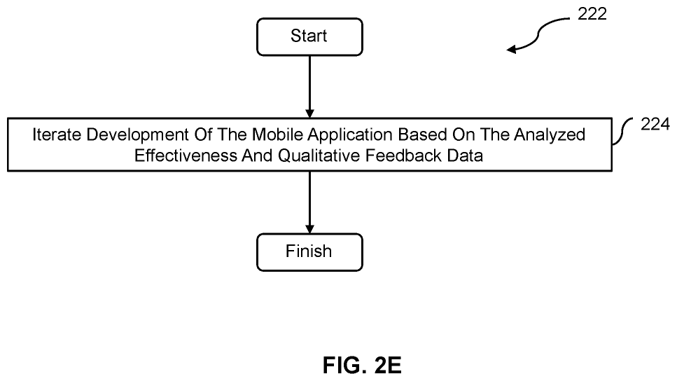

FIGS. 2A, 2B, 2C, 2D and/or 2E illustrate an example flow diagram of a method 200, according to one embodiment. The method 200 may include developing an interactive mobile application including OMM teaching content; providing access to the mobile application to a plurality of medical students at block 202. The method 200 may include collecting baseline OMM competency data from the plurality of medical students before providing access at block 204. The method 200 may include collecting post-intervention OMM competency data after a defined period of access to the mobile application at block 206. The method 200 may include evaluating an effectiveness of the mobile application by analyzing the baseline and post-intervention competency data at block 208.

In FIG. 2B, the method 200 may be continued at 210, and may further include collecting qualitative feedback data from medical students through interviews and focus groups regarding experiences using the mobile application at block 212.

In FIG. 2C, the method 200 may be continued at 214, and may further include tracking usage analytics of interactions with the mobile application by the plurality of medical students at block 216.

In FIG. 2D, the method 200 may be continued at 218, and may further include identifying barriers and facilitators to adoption of the mobile application based on qualitative feedback data at block 220.

In FIG. 2E, the method 200 may be continued at 222, and may further include iterating development of the mobile application based on the analyzed effectiveness and qualitative feedback data at block 224.

In some cases, the method 200 may be performed by one or more hardware processors, such as the processors 126 of FIG. 1, configured by machine-readable instructions, such as the machine-readable instructions 106 of FIG. 1. In this aspect, the method 200 may be configured to be implemented by the modules, such as the modules 108, 110, 112, 114, 116, 118, 120 and/or 122 discussed above in FIG. 1.

In one aspect, the invention provides a method for developing and evaluating an OMM educational mobile app to enhance integration into medical school curricula.

In another aspect, the invention utilizes an app with multimedia content and interactives to augment OMM learning beyond standard lectures.

In one aspect, the invention collects pre- and post-intervention quantitative competency data to assess the app's effectiveness.

In another aspect, the invention conducts qualitative assessments through focus groups and interviews to gain insights into user perspectives.

In one aspect, the invention tracks usage analytics to understand learner engagement with different app features.

In another aspect, the invention identifies adoption barriers and facilitators to optimize integration strategies.

In one aspect, the invention enables integration with electronic medical records to surface relevant OMM information at the point-of-care.

In another aspect, the invention provides a validated pedagogical framework for technology-enabled propagation of declined OMM skills in medical education.

In one aspect, the mobile application focuses specifically on building osteopathic manipulative medicine skills across core specialties, filling a major gap left open by generic medical education apps. Instructional content and features exclusively focus on propagating declining OMM competencies through technology.

In another aspect, detailed treatment demonstration videos are organized by chief complaint presentations across major specialties like emergency medicine and family medicine. This specialty-aligned video catalog systematizes OMM learning by clinical rotation.

In one aspect, video tutorials are supplemented with scientific references and research reinforcing the techniques, helping instill student confidence in how and why OMM helps featured complaints.

In another aspect, experienced osteopathic clinicians provide contextual guidance on clinical diagnosis and reasoning to apply demonstrated OMM treatments through video commentaries.

In one aspect, unlike crowded video platforms, the app fosters an interactive community experience through exclusive networking features enabling communication with peer osteopathic students and physicians.

In another aspect, personalized educational analytics empower students to self-assess OMM mastery within each specialty while navigating video content. This level of customization surpasses competing apps.

In one aspect, the ability to directly schedule video consultations with app-using osteopathic practitioners augments on-demand content through personalized feedback.

In another aspect, inclusion of billing details for demonstrated OMM procedures assists in translating techniques into practical application during rotations and future practice.

### INDUSTRIAL APPLICATION

The mobile application for enhancing osteopathic manipulative medicine competency developed in this invention provides great value to the professional medical education industry. Specifically, osteopathic medical schools and associated teaching hospitals have a strong industrial need to perpetuate manipulative skills within their distinct branch of medicine. Adoption of this invention's specialized educational app as a supplemental tool integrated into standard curricula greatly improves the ability of osteopathic institutions to address long-standing integration obstacles. Its unique pedagogical approach effectively propagates declined techniques to strengthen this key facet of holistic care that differentiates the osteopathic philosophy. This invention' s industrial application cultivates the future osteopathic workforce' s hands-on patient care abilities.

What is claimed is:

1. A method for enhancing clinical readiness among osteopathic medical students, the method comprising:
assessing needs by surveying osteopathic clinicians across medical specialties to identify frequently assessed chief complaints;
planning instructional content by analyzing existing curricula and collaborating with experienced physicians to determine relevant content for the identified chief complaints;
developing a mobile application with a user friendly interface and capabilities for video interaction;
producing instructional videos demonstrating clinical skills for the planned content utilizing simulations or real-life scenarios;
integrating content within the mobile application organized by core medical specialties;
beta testing the mobile application and gathering usability, relevance and satisfaction feedback from osteopathic medical students;
improving the mobile application based on the gathered feedback data;
launching the mobile application including press releases and social media promotion; and
planning regular content updates to keep instructional videos and information current.

2. The method of claim 1, wherein the instructional videos are tailored to address specific clinical skills and knowledge required for each of the specialties.

3. The method of claim 1, further comprising providing a user interface that allows users to access and navigate the instructional videos based on their individual educational needs and preferences.

4. The method of claim 1, wherein the mobile application includes a feature for tracking and recording user progress within each core specialty, enabling personalized learning experiences.

5. The method of claim 1, wherein the mobile application includes the ability to network via video chat, text messaging with other physicians that download the mobile application.

6. The method of claim 1, wherein the instructional videos demonstrate osteopathic manipulative techniques organized by chief complaints within core specialties.

7. The method of claim 1, further comprising collecting qualitative feedback data from medical students through interviews and focus groups regarding experiences using the mobile application.

8. The method of claim 1, further comprising tracking usage analytics of interactions with the instructional videos and other features of the mobile application by the users.

9. The method of claim 1, further comprising iterating development of the mobile application based on analyzed user feedback data to enhance the user experience.

10. A system, comprising:
one or more hardware processors configured by machine-readable instructions to:
assessing needs by surveying osteopathic clinicians across medical specialties to identify frequently assessed chief complaints;
planning instructional content by analyzing existing curricula and collaborating with experienced physicians to determine relevant content for the identified chief complaints;
developing a mobile application with a user friendly interface and capabilities for video interaction;
producing instructional videos demonstrating clinical skills for the planned content utilizing simulations or real-life scenarios;
integrating content within the mobile application organized by core medical specialties;
beta testing the mobile application and gathering usability, relevance and satisfaction feedback from osteopathic medical students;
improving the mobile application based on the gathered feedback data;
launching the mobile application including press releases and social media promotion; and
planning regular content updates to keep instructional videos and information current.

11. The system of claim 10, wherein the instructional videos are tailored to address specific clinical skills and knowledge required for each of the specialties.

12. The system of claim 10, wherein the one or more hardware processors are further configured to provide a user interface that allows users to access and navigate the instructional videos based on their individual educational needs and preferences.

13. The system of claim 10, wherein the mobile application includes a feature for tracking and recording user progress within each core specialty, enabling personalized learning experiences.

14. The system of claim 10, wherein the mobile application includes the ability to network via video chat, text messaging with other physicians that use the mobile application.

15. The system of claim 10, wherein the instructional videos demonstrate osteopathic manipulative techniques organized by chief complaints within core specialties.

16. The system of claim 10, wherein the one or more hardware processors are further configured to collect qualitative feedback data from medical students through interviews and focus groups regarding experiences using the mobile application.

17. The system of claim 10, wherein the one or more hardware processors are further configured to track usage analytics of interactions with the instructional videos and other features of the mobile application by the users.

18. The system of claim 10, wherein the one or more hardware processors are further configured to iterate development of the mobile application based on analyzed user feedback data to enhance the user experience.
